(19) United States
(12) Patent Application Publication
(10) Pub. No.: US 2026/0306124 A1
(43) Pub. Date: Oct. 8, 2026

(54) SKIN TREATMENT DEVICE

(71) Applicant: DD Karma LLC d/b/a Dermaflash, Chicago, IL (US)

(72) Inventor: Dara Levy, Highland Park, IL (US)

(73) Assignee: DD Karma LLC d/b/a Dermaflash, Chicago, IL (US)

(21) Appl. No.: 19/639,725

(22) Filed: Apr. 6, 2026

Related U.S. Application Data
(60) Provisional application No. 63/785,300, filed on Apr. 8, 2025.

(51) Int. Cl. A61B 17/32; A61B 17/00; A61B 17/3213; A61B 17/3215; H02J 7/70

(52) U.S. Cl. CPC A61B 17/320068; A61B 2017/00752; A61B 2017/320075; A61B 17/3213; A61B 17/3215; H02J 7/731

(57) ABSTRACT

Aspects disclosed herein are related to dermaplaning kits comprising dermaplaning devices and other peripheral components. A user may sweep the dermaplaning device over the skin in order to exfoliate the skin. The dermaplaning device may include a device handle configured to selectively engage a corresponding blade cartridge, where the cartridge may be removed and replaced with a new cartridge after being sufficiently used. The peripheral components used in the dermaplaning kit may include a blade rack configured to store blade cartridges, a blade rack holder, a dermaplaning device receptacle, and a blade disposal unit, among other components.

### CROSS-REFERENCE TO RELATED APPLICATIONS

This application claims the benefit under 35 U.S.C. § 119(e) to U.S. Provisional Application Ser. No. 63/785,300, filed on Apr. 8, 2025, which is hereby incorporated by reference in its entirety.

### FIELD

The field of the invention relates to handheld devices and processes used in treating facial skin and more particularly to handheld dermaplaning devices for exfoliating facial skin.

### BACKGROUND

Various processes are known for treating facial skin. One such process is dermaplaning.

Dermaplaning is a relatively popular process used for exfoliating the epidermis, (outermost layer of the skin), and removing fine vellus hair, also called peach fuzz, from the skin. Dermaplaning is a process normally performed by licensed skin care professionals, such as estheticians, who use a scalpel or similar blade. Using a delicate touch, the blade is swept across the skin with light feathering strokes to exfoliate the skin, which involves the removal of the oldest dead skin cells on the skin's outermost surface.

Dermaplaning facial skin has many benefits. For example, removing epidermal skin allows skin care products to penetrate more readily into deeper layers of the skin for better results. As mentioned above, dermaplaning removes vellus hair which tends to cause a build-up of dirt and oils in the follicles. Removal of the hair results in healthier looking skin.

### SUMMARY

According to some embodiments, a dermaplaning kit is provided. The dermaplaning kit may include a base tray and charging base configured to receive and charge a dermaplaning device. The kit may also include a blade cartridge storage rack supported by the base tray, where the rack is configured to hold a plurality of blade cartridges for attachment to the dermaplaning device. The kit may also include a blade cartridge disposal unit supported by the base tray, where the disposal unit is configured to receive and hold discarded blade cartridges.

According to some embodiments, a dermaplaning kit is provided. The dermaplaning kit may include a dermaplaning device with a handle and at least one blade cartridge attachable to the handle. In some embodiments, the dermaplaning device may include a handle extending lengthwise in a longitudinal direction with a head at an end portion of the handle, where the head is configured to attach the at least one blade cartridge to the handle. In some embodiments, the dermaplaning device may also generate vibrations at the head. The dermaplaning kit may also include a charging base with a charging cavity configured to receive and charge the dermaplaning device. In some embodiments, the charging cavity has a shape corresponding to a shape of the dermaplaning device. In some embodiments, the charging cavity includes one or more electrical contacts which align with one or more electrical contacts on the handle when the dermaplaning device is placed in the charging cavity.

According to some embodiments, a dermaplaning kit is provided. The dermaplaning kit may include a base tray configured to receive and store a dermaplaning device and a blade cartridge rack holder supported by the base tray. The blade cartridge rack holder may be configured to hold a plurality of blade cartridge storage racks, and each blade cartridge storage rack may be configured to hold a plurality of blade cartridges for attachment to the dermaplaning device.

According to some embodiments, a dermaplaning device is provided. The dermaplaning device may include a blade cartridge with a blade and a handle extending lengthwise in a longitudinal direction. The handle may include a head at an end portion of the handle, and the head may be configured to releasably attach the blade cartridge to the handle. The dermaplaning device may also include a catch movable between a locked position and an unlocked position. In some embodiments, the catch is configured to retain the blade cartridge at the head when moved to the locked position and to release the blade cartridge from the head when moved to the unlocked position. The dermaplaning device may also include an actuator coupled to the catch, where the actuator is configured to move the catch to the unlocked position when actuated.

According to some embodiments, a blade cartridge for a dermaplaning device is provided. The blade cartridge may include a blade, a housing attached to the blade, and a safety cage configured to be attached to the blade. In some embodiments, the safety cage is configured to limit a depth at which the blade contacts a user's skin. In some embodiments, the safety cage comprises a plurality of projections which extend from a side of the blade to the blade edge. In some embodiments, the plurality of projections each have a tapered shape such that a width of a proximal portion of the projections is greater than a width of a distal portion of the projections.

According to some embodiments, a blade cartridge for a dermaplaning device is provided. The blade cartridge may include a blade, a housing attached to the blade, and a mounting flange. In some embodiments, the mounting flange may include a first pair of latches configured to engage a corresponding second pair of latches on a dermaplaning device to attach the blade cartridge to the dermaplaning device.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a perspective view of a dermaplaning kit, according to some embodiments;

FIG. 2A is a front perspective view of a dermaplaning device without an attached blade cartridge, according to some embodiments;

FIG. 2B is a rear perspective view of the dermaplaning device of FIG. 2A with an attached blade cartridge, according to some embodiments;

FIG. 2C is a bottom view of the dermaplaning device of FIG. 2B;

FIG. 3 is a cross-sectional perspective view of a head of the dermaplaning device of FIG. 2A, according to some embodiments;

FIG. 4 is a side view of a blade cartridge configured to engage a dermaplaning device, according to some embodiments;

FIG. 5 is a cross-sectional perspective view of a head of the dermaplaning device of FIG. 2B, according to some embodiments;

FIG. 6A depicts a bottom view of a dermaplaning device, according to some embodiments;

FIG. 6B depicts a top view of a blade cartridge configured to be engaged with the dermaplaning device of FIG. 6A, according to some embodiments;

FIG. 7A is a cross-sectional view of a blade cartridge housing, according to some embodiments;

FIG. 7B is a cross-sectional view of a blade cartridge housing, according to some embodiments;

FIG. 7C is a side view of a blade partially covered by a safety cage, according to some embodiments;

FIG. 7D is an enlarged view of the arrangement of FIG. 7C;

FIG. 8A is an enlarged view comparing two blade and safety cage arrangements, according to some embodiments;

FIG. 8B depicts a view of one of the blade and safety cage arrangements of FIG. 8A subject to deformation during use, according to some embodiments;

FIG. 9A is a front perspective view of a blade rack, according to some embodiments;

FIG. 9B is a rear perspective view of the blade rack of FIG. 9A shown in combination with a dermaplaning device arranged to receive a cartridge in the blade rack, according to some embodiments;

FIG. 9C is a bottom view of the blade rack of FIG. 9A;

FIG. 9D is an enlarged view of the blade rack of FIG. 9A;

FIG. 9E is an enlarged cross-sectional view of the blade rack of FIG. 9A depicting a blade cartridge being received in a channel of the blade rack, according to some embodiments;

FIG. 10A is a perspective view of a blade rack holder, according to some embodiments;

FIG. 10B is a front view of the blade rack holder of FIG. 10A,

FIG. 11 is a top view of a dermaplaning device holder, according to some embodiments;

FIG. 12A is a perspective view of a blade disposal unit, according to some embodiments;

FIG. 12B is a perspective view of the blade disposal unit of FIG. 12A with the lid removed, according to some embodiments;

FIG. 12C is a perspective view of the lid of the blade disposal unit of FIG. 12A, according to some embodiments;

FIG. 13 is a perspective view of a base tray configured to receive various components of a dermaplaning kit, according to some embodiments; and

FIG. 14 is a view of a dermaplaning device arranged in relation to a user's skin at an angle, according to some embodiments.

### DETAILED DESCRIPTION

Conventional hand-held devices used for dermaplaning normally include a surgical style scalpel consisting of a blade and a handle. Such scalpels are intended for use only in licensed medical spas, not for use in non-medical resort spas or day spas. Additionally, the inventor(s) have recognized that such scalpels may be inconvenient to use even in licensed medical spas, as the handles are often narrow and uncomfortable to hold. Such scalpels are also generally do not include changeable blades, so that the entire scalpel must be disposed of after use. Such scalpels also do not include any vibration functionality, which may improve the smoothness of skin upon completion of dermaplaning.

The inventor(s) have also appreciated that, while handheld dermaplaning devices for personal use are known (some of which have vibration functionality), such devices are not particularly suitable for use in professional spas. Home use dermaplaning devices are intended for intermittent use, as opposed to frequent use throughout a given day as would be expected for use in professional spas. As a result, these home use dermaplaning devices are not intended for continuous and sanitary replacement of blade cartridges, for sanitary disposal of multiple used blade cartridges, for convenient storage of a large number and/or variety of types of blade cartridges, for rapid and convenient recharging, or for other efficiency related features which are beneficial for providing dermaplaning services at a resort/day spa.

As a result, the inventors have recognized a need for a dermaplaning kit which provides storage for a relatively large supply of replacement blade cartridges and convenient disposal of used blade cartridges. The inventors have also recognized a need for convenient recharging of the dermaplaning device when the device is not in use. The inventors have also recognized a need for a method of attaching and detaching blades to a handheld device.

The present disclosure is directed to a dermaplaning kit for dermaplaning skin and a method of using the dermaplaning kit.

In some aspects, the dermaplaning kit may include a handheld dermaplaning device to allow a user to perform dermaplaning. The device may include a blade which the user may sweep over skin in order to exfoliate the skin. The device may also include a handle having an ergonomic shape in order to allow for comfortable and precise use of the device.

In some aspects, it may be desirable for the device to vibrate while being used. Such vibrations may move the blade in a “wave-like” pattern over the skin in order to resurface the skin and provide for a smoother glide of the blade over the skin. The vibration may be generated by a motor, such as a sonic motor, mounted in the device handle. A battery may be provided in the device handle to power the motor. In some embodiments, the battery is a replaceable battery. In other embodiments, the battery is a rechargeable battery.

In some aspects, it may be desirable to allow the blade to be detachable from the handle and for a different blade to be attached to the handle. For example, this may be desirable if a blade becomes dull, or if the user wishes to use a different style of blade, or if the user is using the device on multiple people and wishes to avoid cross-contamination. Therefore, the blade may be part of a blade cartridge, which may be selectively attached to the handle. The blade cartridge may include a blade housing which holds the blade, and allows the blade cartridge to be attached to the device handle.

In some aspects, it may be desirable to use different styles of blades. For example, a blade may be equipped with a safety cage which limits the depth the blade may penetrate into the skin. However, licensed medical professionals and other users may prefer to use a blade without such a safety cage. The dermaplaning kit may therefore include blade cartridges with safety cages and blade cartridges without safety cages.

In some aspects, it may be desirable to store multiple blade cartridges together in a single component in order to allow for efficient and organized changing, transport, and storage of blades. The dermaplaning kit may therefore include a blade rack for the storage of a plurality of blade cartridges.

In some aspects, it may be desirable for the blade rack to be configured to allow a user to attach a blade cartridge to the device handle without the user needing to touch the blade cartridge with their hands. The blade rack may be configured to hold the blade cartridges with the blade housing exposed so that a user simply must press the device handle onto the blade cartridge in order to attach the blade cartridge to the handle. More details on how the blade cartridge is attached to the device handle are included below. In some aspects, the blade rack may be configured to lightly grasp the blade cartridges in order to prevent blade cartridges from falling out when the blade rack is moved, but still allowing them to be removed by the user when desired.

In some aspects, it may be desirable for the dermaplaning kit to allow for the storage of multiple blade racks. This could allow the user to store multiple different styles of blades, or simply store a larger quantity of blade cartridges. The dermaplaning kit may therefore include a blade rack storage unit configured to hold multiple blade racks. In some aspects, the blade rack storage unit may be configured to allow the blade racks to slide into the blade rack storage unit at different levels so that each blade rack may be accessed without moving the other blade racks. In some aspects, the blade racks may include tabs which a user may grasp in order to remove the blade racks from the blade rack storage unit.

In some aspects, the dermaplaning kit may include a device receptacle to hold the device when not in use. If desired, the device receptacle may also be configured to charge the device when the device is placed in the device receptacle, so that the user does not need to remember to charge the device separately. The device receptacle may be configured to hold the device with or without a blade cartridge attached.

In some aspects, it may be desirable for a user to quickly dispose of used cartridges. The dermaplaning kit may therefore include a disposal container for the disposal of used blade cartridges. In some aspects, the disposal container may include a lid to prevent the used cartridges from spilling out if the disposal container is tipped. The lid may have a hole sized to allow blade cartridges to be inserted. This hole may include a cover which may be openable to allow blades to be inserted into the hole, and closable to prevent blades from exiting through the hole if the disposal container is tipped. In some aspects, the lid may be removable in order to allow blade cartridges to be easily dumped from the disposal container.

In some aspects, it may be desirable for the above components to be combined into a single unit in order to allow for easier transport of the dermaplaning kit, and to allow the dermaplaning kit to be organized and aesthetically pleasing. The dermaplaning kit may include a base into which any of the above components may be inserted. In some embodiments, the components may be integrally molded into the base. In other embodiments, the components are permanently or selectively attachable to the base.

It should be understood that any combination of components described above may be included in the dermaplaning kit, and any combination of those components may be integrally molded with the base, permanently attached to the base, or selectively attached to the base. It should also be understood that the base may not be necessary at all in some embodiments, and that the components above may all be separate.

In some aspects, it may be desirable to have a lid which covers at least some of the components attached to the base. A lid may be desirable to increase the aesthetics of the dermaplaning kit, and may also protect the device from dust and falling objects. For example, in one embodiment, the base includes a lid which covers the device, the device receptacle, the blade racks, and the blade rack storage unit.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

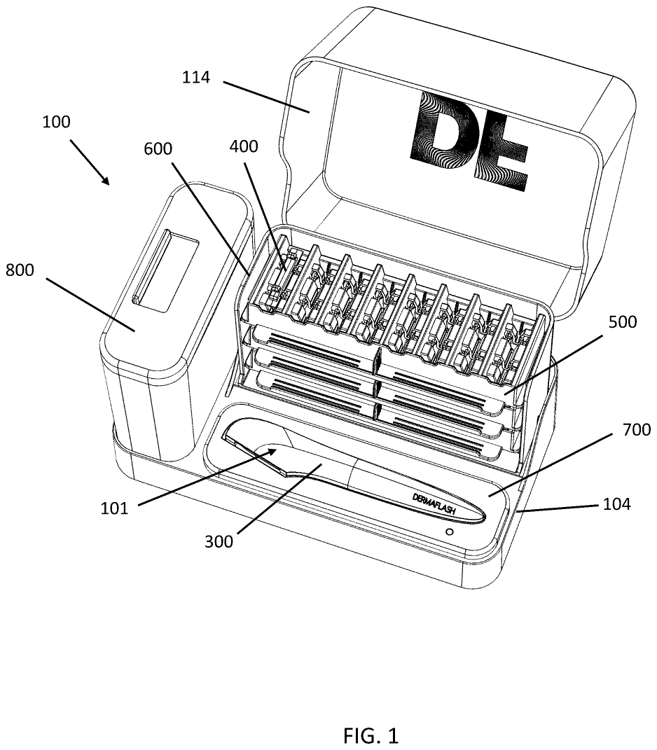

FIG. 1 illustrates an embodiment of a dermaplaning kit 100. The dermaplaning kit 100 may include a dermaplaning device 101, as well as a plurality of peripheral components. As shown in FIG. 1, the dermaplaning device 101 includes a device handle 300 configured to engage a blade cartridge 400. The kit 100 also includes a blade rack 500 configured to store blade cartridges 400, a blade rack holder 600, a dermaplaning device receptacle 700, and a blade disposal unit 800. It should be understood that FIG. 1 is simply an illustrative embodiment of the dermaplaning kit 100, and that the dermaplaning kit may include any number and combination of peripheral components. The dermaplaning device 101 and each of the peripheral components are described in detail below.

### Dermaplaning Device

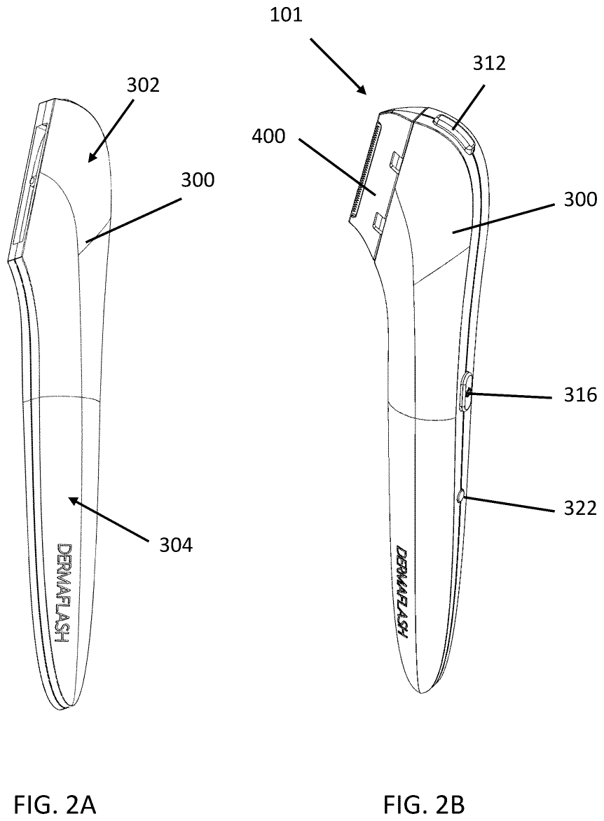

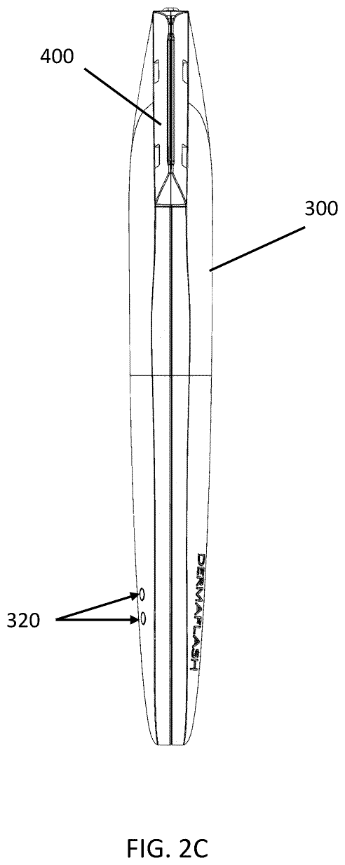

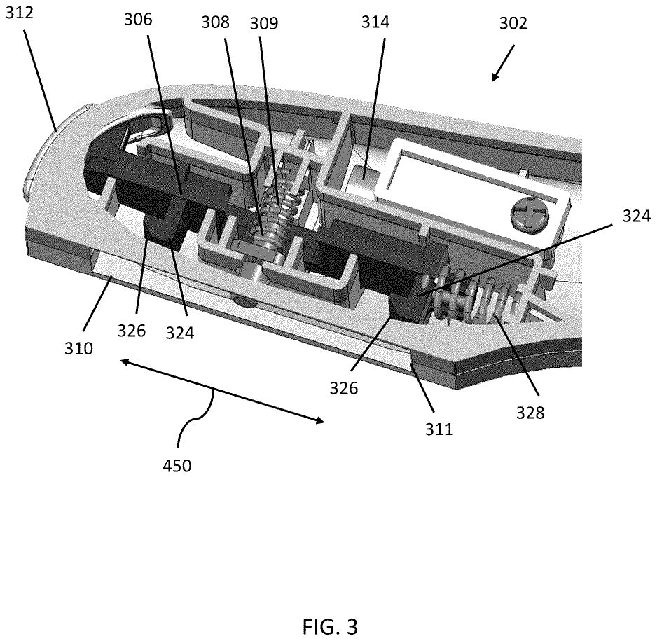

FIGS. 2A-2C illustrate various views of the dermaplaning device 101 according to some embodiments. FIG. 3 shows a cross-sectional perspective view of a device head 302 of the dermaplaning device 101.

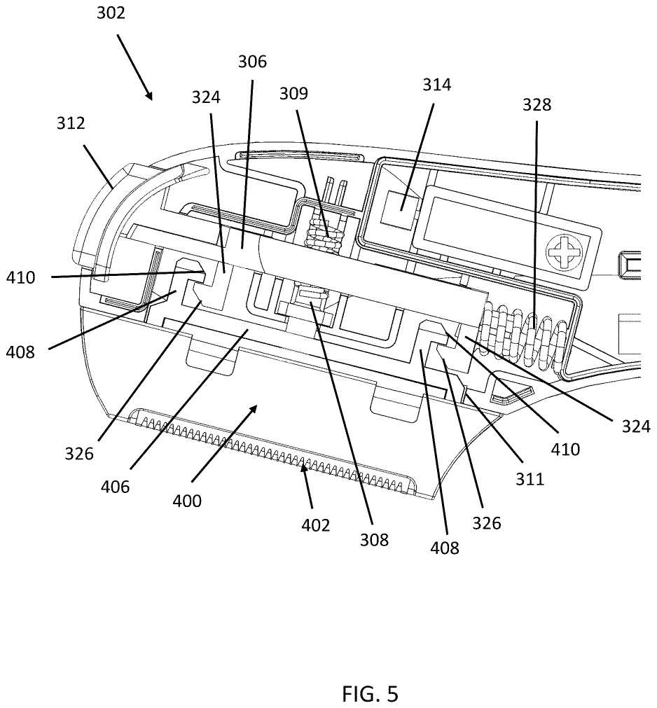

The dermaplaning device 101 may include a device handle 300 configured to engage a blade cartridge 400 having a blade 402 (see FIG. 5). In use, a user grasps the device handle 300 and sweeps blade 402 across the skin in order to exfoliate the skin. The device handle 300 may extend lengthwise in a longitudinal direction such that the device includes a device head 302 and a tail portion 304.

In some embodiments, the device handle 300 includes a motor 314, such as a sonic motor, to vibrate the dermaplaning device 101 while in use. Such vibrations may allow the blade 402 to move in a “wave-like” fashion to glide more easily over the skin, thereby resulting in a smoother skin surface upon completion than would result without such vibrations. The motor 314 may be mounted in the device head 302 so that the motor is close to the blade 402 (see FIG. 5). This proximity to blade 402 allows vibrations from the motor 314 to be more effectively transferred to the blade 402 than if the motor was mounted further from the blade 402. However, it should be understood that the motor 314 may be located anywhere in the device handle 300 or may not be included at all in some embodiments.

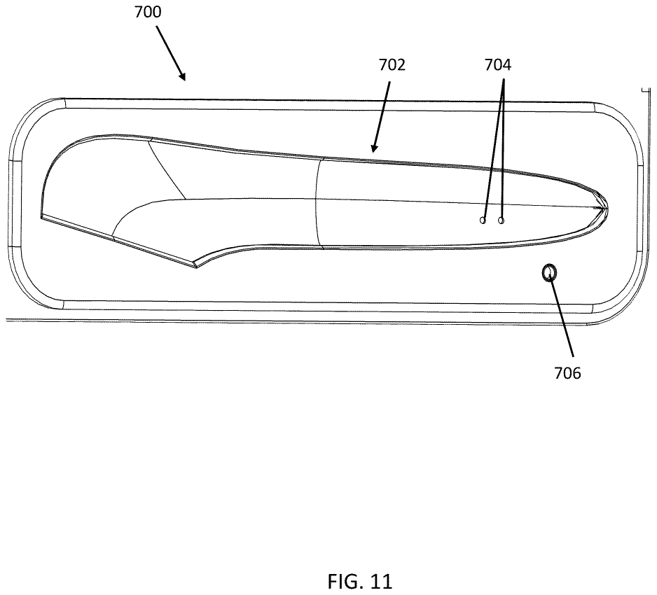

In some embodiments, the motor 314 may be powered by a battery located within the device handle 300. For example, the battery may be mounted within a tail portion 304 of the device. In some embodiments, the battery may be a replaceable battery. In other embodiments, the battery is a rechargeable battery. In some embodiments, when the battery is a rechargeable battery, the dermaplaning device may include device charging contacts 320 to allow for charging of the battery. In some embodiments, the charging contacts 320 are located in the tail portion 304 and extend through an exterior casing of the device handle 300 as shown in FIG. 2C. However, it should be understood that the charging contacts 320 may be located anywhere inside the device handle 300. The charging contacts may be configured to align with charging contacts 704 on the device receptacle 700, as shown in FIG. 11 and described below.

In some embodiments, it may be desirable to turn vibrations on or off, or adjust the intensity of vibrations according to user preference. Accordingly, the device handle 300 may include at least one power button 316, as shown in FIG. 2B. The power button 316 may be mounted to any portion of the device handle 300. The power button 316 may turn the motor 314 on and off, and may adjust the intensity of vibrations from the motor 314. While the illustrated embodiment shows a single button 316 which is configured to turn the motor 314 on and off and adjust the intensity of vibrations, it should be understood that multiple buttons could be used to perform these functions. It should also be understood that such buttons may be any suitable type of button, such as capacitive touch buttons, push buttons, or toggle switches.

In some embodiments, the dermaplaning device 101 may have an indicator light 322 mounted on the device handle 300. This indicator light may change in color, brightness, and pattern in order to provide a user with information about device status. For example, when the motor 314 is off, indicator light 322 may be off as well. When the vibration motor 314 is set to a low intensity, the indicator light 322 may turn on at a low brightness level. When the vibration motor 314 is set to a high intensity, the indicator light 322 may turn on at a high brightness. It should be understood that the above examples are simply illustrative embodiments and that any combination of color, pattern, or brightness may be used to indicate any device status information.

### Blade Cartridge Attachment

In some embodiments, it may be desirable for the blade cartridge 400 to be removably attachable to the device handle 300, such that multiple different blade cartridges 400 may be attachable to the device handle 300. For example, a user may switch to a new blade when the current blade becomes dull. Also, if a user performs dermaplaning services for various clients, the user may want to switch blades for each client to prevent cross-contamination. It may also be desirable to use different styles of blades with the dermaplaning device 101.

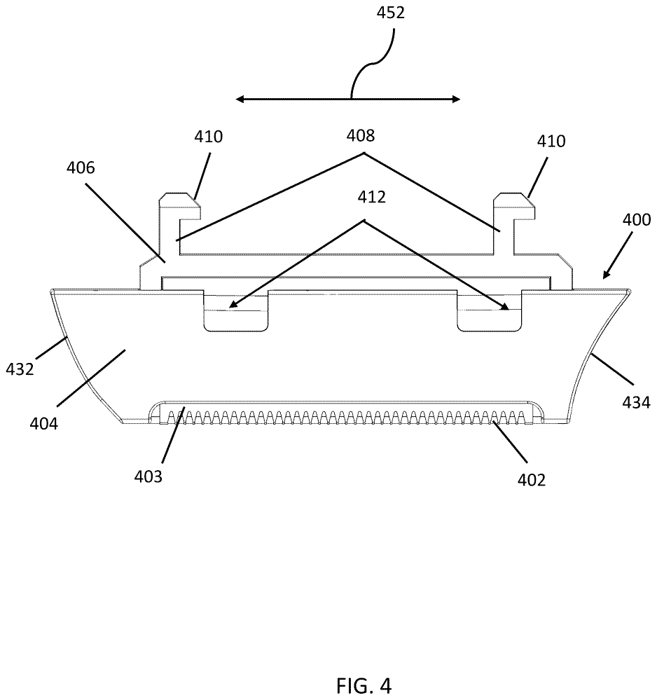

FIG. 4 illustrates a side view of the blade cartridge 400 detached from the device handle 300. FIG. 5 illustrates the device head 302 with the blade cartridge 400 attached.

In some embodiments, the blade cartridge 400 includes a mounting flange 406 (see FIG. 4), which may be inserted into aperture 310 (see FIG. 3) on the device head 302. In some embodiments, the blade cartridge 400 is locked into the device head 302 via corresponding fasteners in the device head 302 and on the mounting flange 406. In particular, the device head 302 may include a catch 306. The catch 306 may include device hooks 324 spaced apart in a first direction 450 (see FIG. 3), which are configured to selectively grasp corresponding blade cartridge hooks 408 spaced apart in a second direction 452 (see FIG. 4) on the mounting flange 406. When the device hooks 324 are engaged with blade cartridge hooks 408 such that the blade cartridge 400 is mounted to the device head 302 (see FIG. 5), the first direction may be parallel to the second direction. In some embodiments, the device hooks 324 may be spaced apart at a first distance while the blade cartridge hooks 408 are spaced apart at a second distance. In some embodiments, the first distance is equal to the second distance. In other embodiments, however, the first and second distances are unequal. While FIG. 5 shows 2 sets of corresponding hook pairs, it should be understood that any number of hooks may be used. Although device hooks 324 and blade cartridge hooks 408 are described above, any suitable type of latches may be provided as the disclosure is not so limited.

In some embodiments, the catch 306 has a locked position, where the device hooks 324 grasp blade cartridge hooks 408, and an unlocked position, where the device hooks 324 do not grasp blade cartridge hooks 408. In some embodiments, the catch 306 is biased into the locked position via a biasing member 328 (e.g. a spring or other resilient member).

In order to attach a blade cartridge 400 to device head 302, the catch 306 must first be moved to the unlocked position. In some embodiments, the catch 306 may be moved into the unlocked position by depressing button 312, which may move the device hooks 324 along a first direction (e.g., the direction along which the hooks 324 are spaced apart). The mounting flange 406 may then be inserted into the aperture 310, and the button 312 may be released. Once the button 312 is released, the biasing member 328 may move the catch 306 into the locked position so that the device hooks 324 grasp blade cartridge hooks 408. Although a button 312 is shown in FIG. 3 and FIG. 5, any suitable actuator may be used to move the catch 306 between the locked and unlocked positions when the actuator is actuated as the disclosure is not so limited.

In some embodiments, the blade cartridge 400 may be attached to device head 302 simply by pressing the mounting flange 406 into aperture 310. In this embodiment, the device hooks 324 may include cam surfaces 326, and the blade cartridge hooks 408 may include corresponding cam surfaces 410. When the mounting flange 406 is pressed into the aperture 310, the cam surfaces 410 of the blade cartridge hooks 408 may contact the cam surfaces 326 of the device hooks 324. This contact may push the catch 306 into the unlocked position. Once the mounting flange 406 is fully inserted into the device head 302, the cam surfaces 410 may no longer be in contact with the cam surfaces 326, and the biasing member 328 may push the catch 306 back into the locked position so that the device hooks 324 grasp the blade cartridge hooks 408. In some embodiments, the cam surfaces 326, 410 may be constructed and arranged as chamfered surfaces, as shown in FIG. 5.

In order to detach the blade cartridge 400 from the device head 302, button 312 may be depressed to move the catch from the locked position to the unlocked position. The contact between mounting flange 406 and aperture edge 311 prevents the blade cartridge hooks 408 from moving with the device hooks 324. Thus, when the catch 306 moves into the unlocked position, the device hooks 324 release blade cartridge hooks 408, allowing the blade cartridge 400 to be removed from device head 302.

In some embodiments, device head 302 may include an ejector, e.g., an ejection plunger 308 (see FIG. 3 and FIG. 5) which may force the blade cartridge 400 out of the device head 302 when the catch 306 is in the unlocked position. This may be desirable to ensure that the blade cartridge 400 exits device the head 302 reliably when the button 312 is depressed. In some embodiments, ejection plunger 308 may include ejection plunger biasing member 309 (e.g. a spring or other resilient member), which biases ejection plunger 308 into a first, extended position. When the blade cartridge 400 is inserted into the device head 302, the mounting flange 406 may push the ejection plunger 308 into a second, compressed position, and the ejection plunger 308 may be held in the compressed position when the corresponding hooks grasp each other. When the corresponding hooks are moved out of alignment, the ejection plunger biasing member may push the ejection plunger 308 back to the extended position, which forces the blade cartridge 400 out of the device head 302.

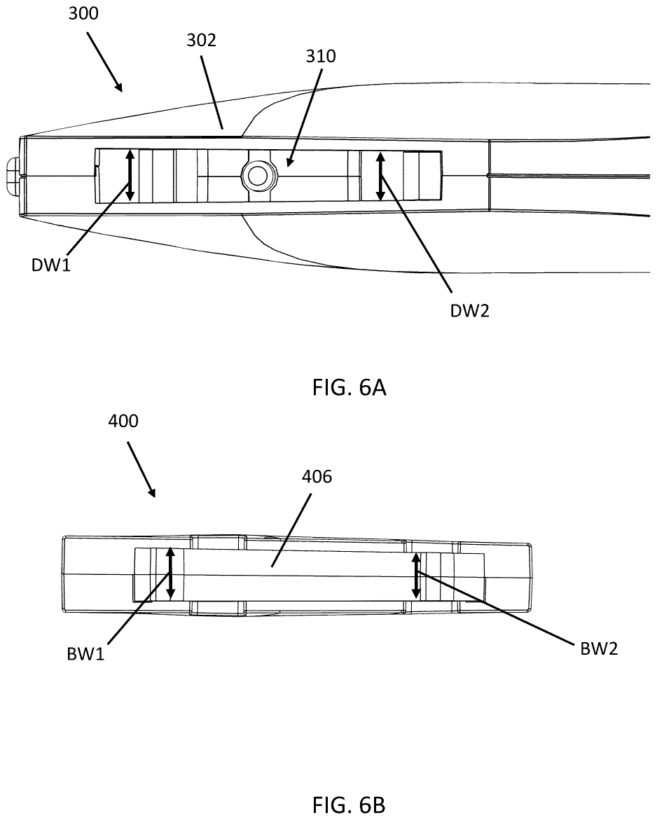

In some embodiments, the aperture 310 and the mounting flange 406 may be shaped such that mounting flange 406 only fits into aperture 310 in a single orientation. FIGS. 6A-6B show an illustrative embodiment of this arrangement. FIG. 6A shows a bottom view of the device head 302 of the device handle 300, where the aperture 310 may have a first width DW1 which is different than a second width DW2. FIG. 6B shows a top view of the blade cartridge 400, where the mounting flange 406 may have a first width BW1 which is different than second width BW2. The first and second widths BW1, BW2 of the mounting flange may correspond to the first and second widths DW1, DW2 of the aperture, such that the mounting flange 406 only fits into aperture 310 when BW1 aligns with DW1, and when BW2 aligns with DW2. In some embodiments, the device hooks 324 and the blade cartridge hooks 408 may only be latched together when the blade cartridge is inserted in a single orientation. For example, the hooks 324, 408 may only grasp one another when the mounting flange 406 is inserted such that the bend of the hooks 324 is oriented towards the bend of the hooks 408 (i.e., the cam surfaces 326 and 410 are oriented towards one another), as shown in FIG. 5.

### Blade Cartridge Structure

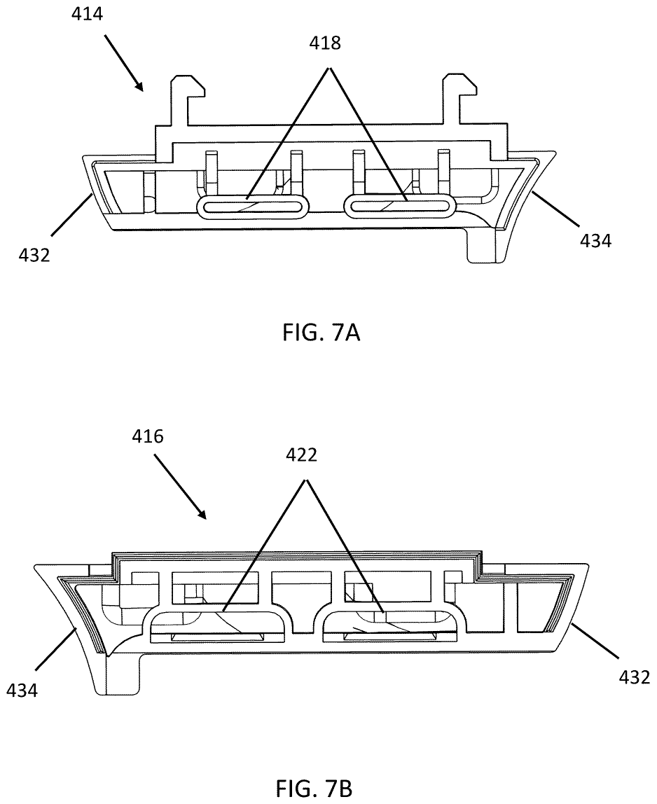

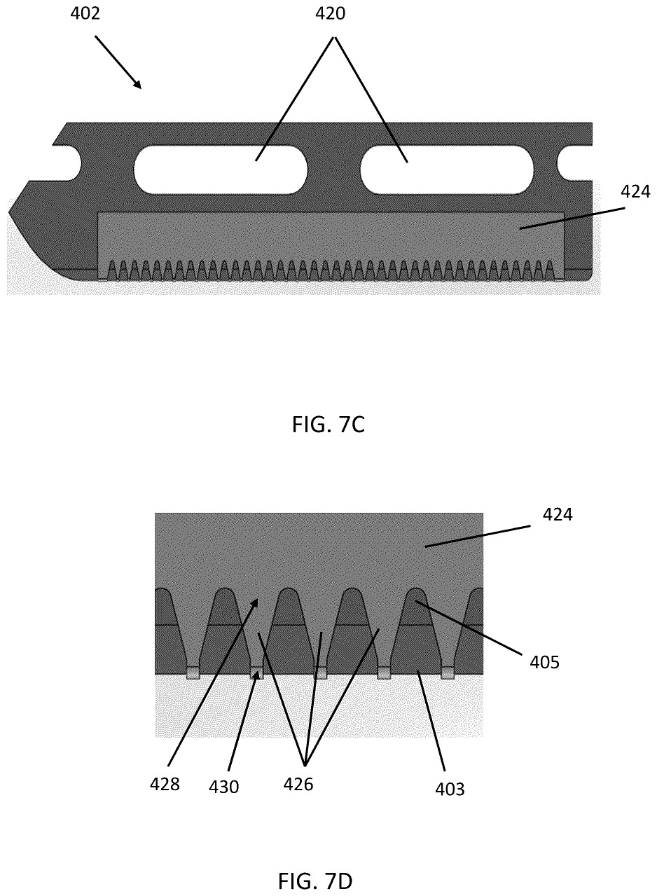

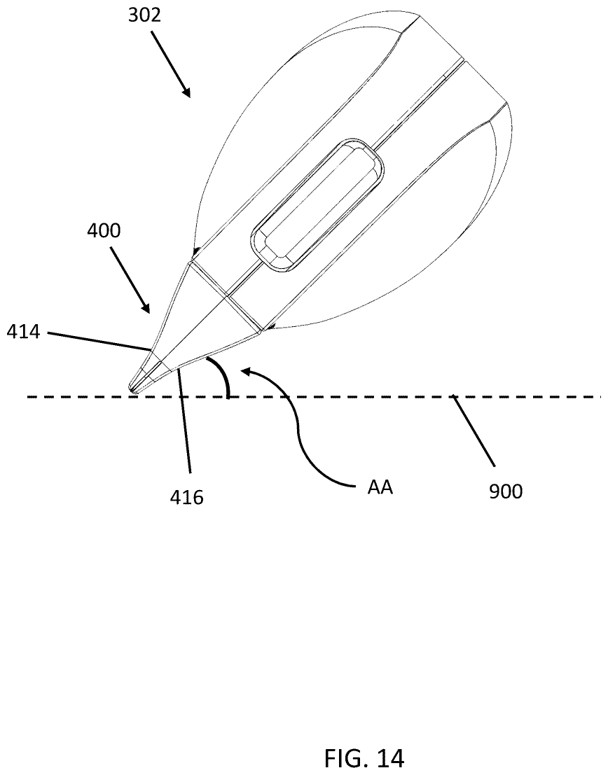

As mentioned above, the blade cartridge 400 may be configured to hold the blade 402. FIGS. 7A-7D show various views of a disassembled blade cartridge 400. In some embodiments, the cartridge housing 404 may include a first housing section 414 and a second housing section 416. The blade 402 may be disposed between the first housing section 414 and the second housing section 416. In some embodiments, the cartridge housing 404 may have an external curvature that is shaped in a concave and/or convex fashion. For example, a first region 432 (i.e., a distal “nose” region”) and a second region 434 (i.e., a proximal “tail” region) of the cartridge housing 404 shown in FIG. 14 may be convex and concave, respectively (see FIG. 4 and FIGS. 7A-7B). In other embodiments, however, first region 432 may be concave while the second region 434 is convex. The first and second regions 432, 434 may instead be both concave or both convex, as the disclosure is not so limited.

The first housing section 414 may include projections 418, which correspond to blade grooves 420 in the blade 402 and to housing section grooves 422 in the second housing section 416. When assembled, the projections 418 may pass through the blade grooves 420 and interlock with the housing section grooves 422. In this manner, the blade 402 may be held securely inside of the assembly blade housing 404. The blade housing sections 414 and 416 may be held together using any suitable fasteners, such as locking tabs, adhesive, or screws.

In some embodiments, the blade 402 may include a safety cage 424. The safety cage 424 may be configured to limit the maximum cutting depth of the blade 402 (i.e., a depth at which the blade may contact or penetrate the user's skin). In some embodiments, safety cage 424 may include a plurality of projections or ribs 426 extending from a side 405 of the blade 402 to the blade edge 403. The ribs 426 may be spaced along a longitudinal direction of the blade 402 such that portions of the blade edge 403 are exposed. In some embodiments, the spacing of ribs 426 is substantially identical, such that an identical amount of blade edge 403 is exposed between each rib 426. In other embodiments, however, the spacing of ribs 426 may follow an alternating pattern, such that the amount of blade edge 403 which is exposed between each rib 426 alternates between a larger amount and a smaller amount.

In some embodiments, the safety cage 424 may be constructed and arranged such that the ribs 426 have a tapered shape. That is, a base 428 (i.e., a proximal portion) of the ribs 426 may be wider than a tip 430 (i.e., a distal portion) of the ribs, as shown in FIG. 7D. The inventor(s) have appreciated that such a configuration may increase the amount of exposed edge of the blade 402 while also improving the structural integrity of the safety cage. That is, the wider base 428 may increase the resilience of the ribs 426 to deformation or breakage that may result from mechanical impact and stress during manufacturing or use of the device. Such deformation or breakage may cause potential injury to the user if the safety cage is not replaced. In addition, the inventors have appreciated that constructing the base 428 of the ribs 426 to be wider than the tips 430 may also serve to limit excessive vibrational energy from being delivered to the tip 430 during the manufacturing process used to form the cage around the blade (e.g., ultrasonic welding). Reducing such excess vibrational energy may be desirable as the vibrational energy may otherwise partially or fully break the ribs during manufacturing, thereby potentially leading to injury of the user when they use the blade cartridge. In some embodiments, a ratio between a width of the base 428 and a width of the tip 430 may be approximately 2:1. However, the ratio may instead be 1.5:1, 2:1, 2.5:1, 3:1, 3.5:1, 4:1, 4.5:1, 5:1, or any other suitable ratio as the disclosure is not so limited. In some embodiments, a ratio between the width of the exposed blade edge and the width of the tip 430 of the ribs 426 may be approximately 5:1 in FIG. 7D. However, the ratio may instead be 2:1, 3:1, 4:1, 6:1, 7:1, 8:1, or any other suitable ratio as the disclosure is not so limited.

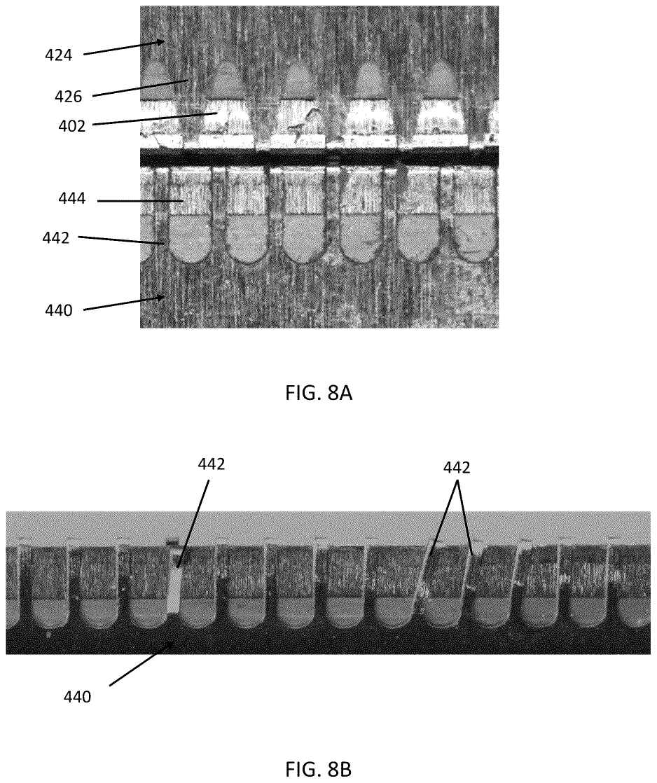

FIG. 8A shows a comparison between first and second embodiments of a blade and safety cage arrangement. Specifically, the first, top embodiment shows a blade 402 partially enclosed by a safety cage 424 having ribs 426 similar to that of FIGS. 7A-7D described above. In contrast, the second, bottom embodiment of FIG. 8A shows a blade 444 partially enclosed by a safety cage 440 having ribs 442 with a thinner base as compared to the top embodiment. FIG. 8B further shows the second embodiment of FIG. 8A after being subjected to continuous use. As shown in FIG. 8B, several of the ribs 442 of safety cage 440 deformed due to their thinner base relative to the ribs 426. Thus, the inventor(s) have appreciated that providing a safety cage having ribs with a wider base may increase the resilience of the ribs to deformation.

### Blade Rack

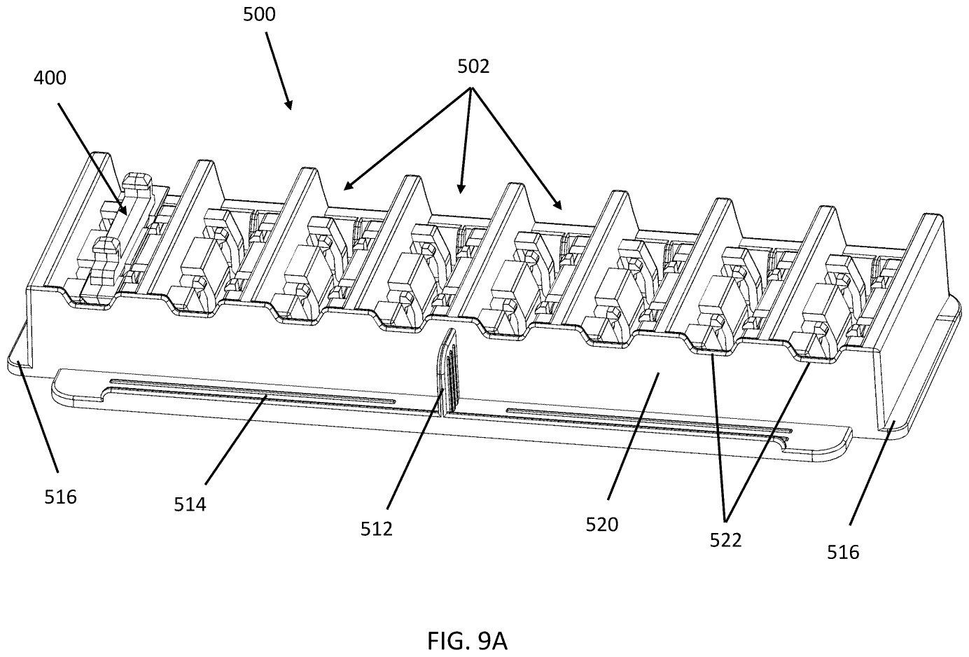

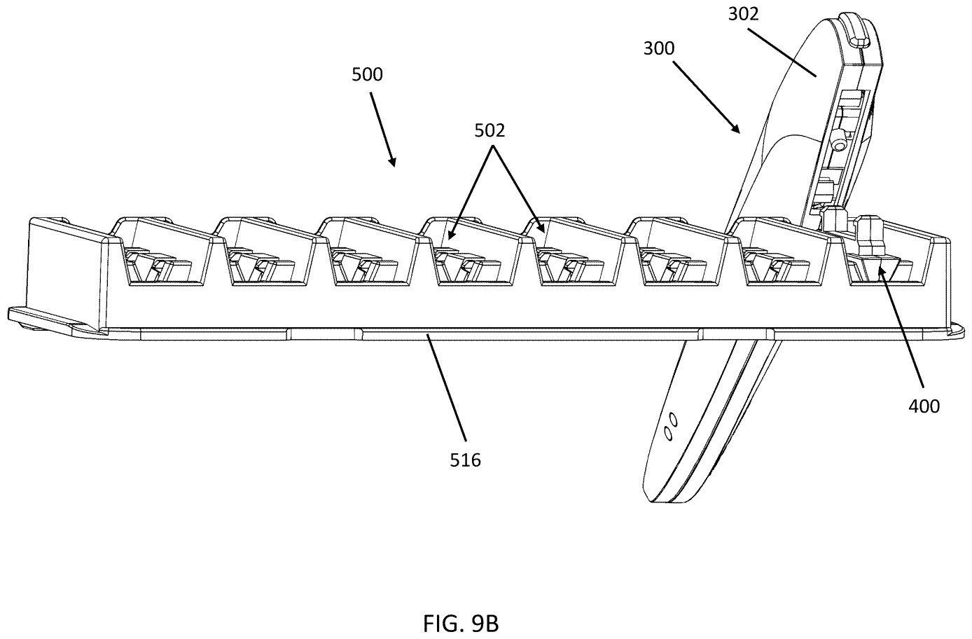

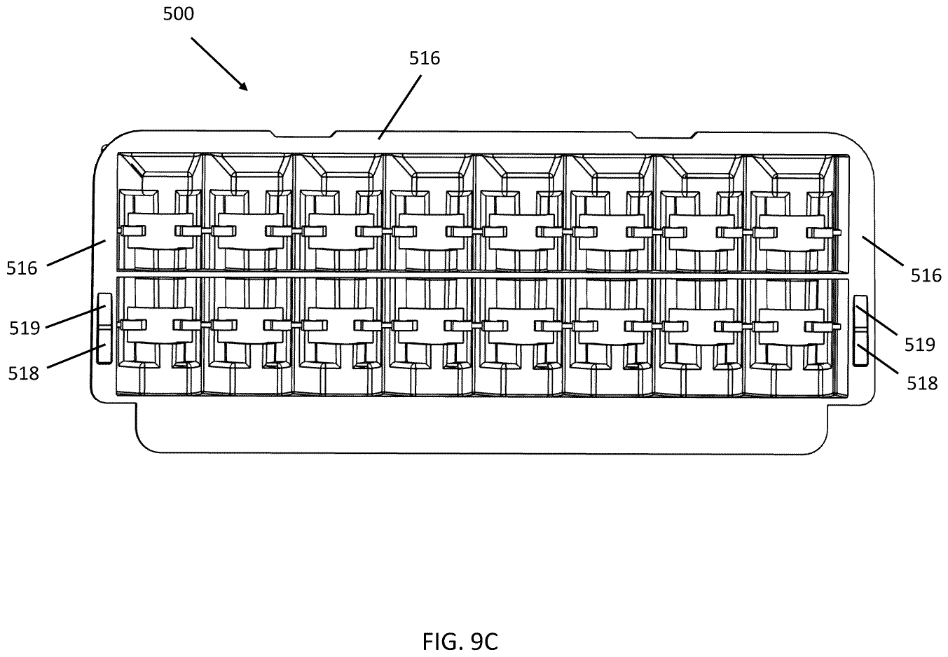

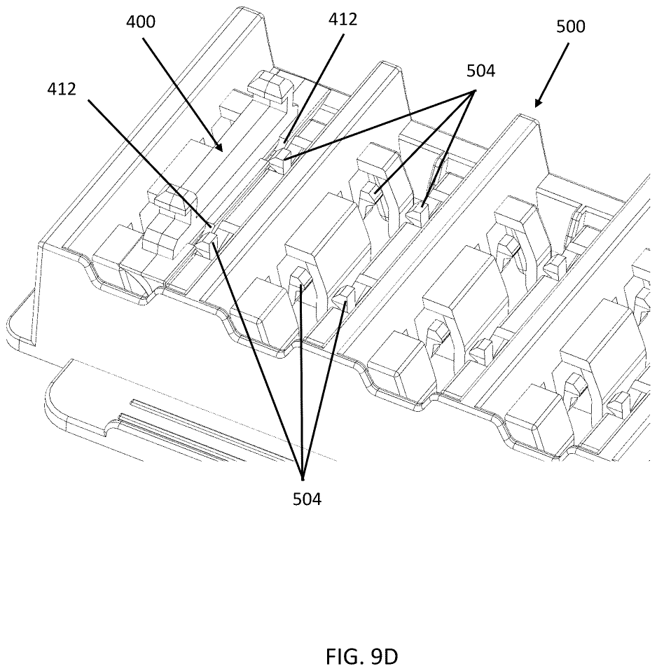

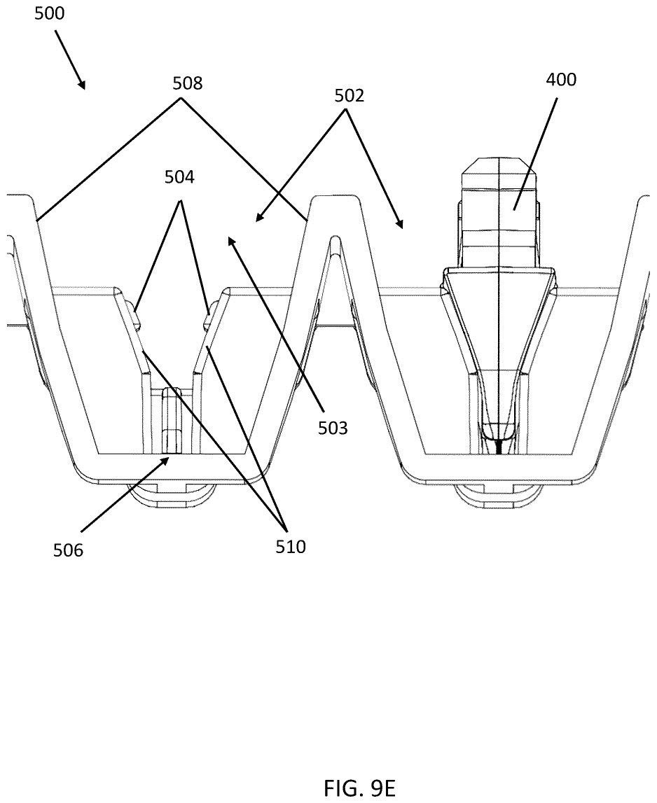

In some embodiments, it may be desirable to include components which allow for organized and convenient storage of unused blade cartridges, so that a user may quickly switch between blade cartridges when desired, and may easily and accurately determine the quantity of unused blade cartridges they have left. In some embodiments, a dermaplaning kit 100 may include at least one blade rack 500 (also referred to as “blade cartridge storage rack” herein) for the storage of blade cartridges 400. FIGS. 9A-9E illustrate various views of a blade rack 500 according to an embodiment.

The blade rack 500 may include any number of slots or channels 502 configured to hold blade cartridges 400. That is, each channel 502 may hold a single blade cartridge 400. In some embodiments, the channels 502 may have an upper opening 503 (see FIG. 9E) such that a user may be able to see if a channel 502 is currently holding a blade cartridge 400. In this manner, a user may easily ascertain the quantity of blade cartridges remaining in the blade rack 500.

In some embodiments, the channels 502 may be configured to grasp the respective blade cartridges 400 such that blade cartridges 400 do not fall out of the channels 502 when blade rack 500 is moved or jostled. In one embodiment, channels 502 include tabs 504 (see FIGS. 9D, 9E) which project into the channel. The tabs 504 may be configured to grasp corresponding grooves 412 in the blade cartridge 400. The tabs 504 may be compliant, such that exerting a sufficient threshold force may bend tabs 504 out of the channel and allow blade cartridges 400 to be inserted or removed from the channel 502. The force necessary to bend tabs 504 may be large enough to prevent blade cartridges 400 from being removed by ancillary forces resulting from movement of the blade racks 500, but small enough that a user may easily pull a blade cartridge 400 out of the respective channel 502 when desired.

In some embodiments, it may be desirable to allow a user to attach a blade cartridge 400 to the device head 302 without the user needing to hold the blade cartridge itself (see FIG. 9B). In this embodiment, channels 502 may be configured to allow a user to attach a blade cartridge 400 to the device head 302 while the blade cartridge 400 is still in the channel 502, and the user may then simply pull on the device handle 300 to remove blade cartridge 400 from blade channel 502. The blade channel 502 may be configured to hold a blade cartridge 400 such that the blade of the blade cartridge 400 faces a bottom surface 506 (see FIG. 9E) of the blade channel 502, and the mounting flange 406 faces the upper opening 503 of the channel. The device head 302 may then be lowered over the mounting flange 406 to attach a blade cartridge 400 to the device handle 300 using any method described above. Channel sidewalls 508 and 510 (see FIG. 9E) may be sized to allow the device head 302 to attach to blade cartridge 400 without interference from the sidewalls. In some embodiments, channel sidewalls 508 and 510 are wide enough to allow the device head 302 to fit between them. In other embodiments, channel sidewalls 508 and 510 may be short enough so that the mounting flange 406 extends above the channel sidewalls 508 and 510. In some embodiments, the channel sidewalls 508 and 510 may be chamfered (as shown in FIG. 9E) to align the blade cartridge 400 within the channel for both loading and unloading of the cartridge.

In some embodiments, such as in FIG. 9A, the blade rack 500 may include a vertical grasping tab 512 and a horizontal grasping tab 514 to allow a user to grasp and move the blade rack 500 more easily. It should be appreciated that any number of grasping tabs 512 and 514 may be used, and that a blade rack 500 may include only vertical grasping tab 512 or horizontal grasping tab 514.

In some embodiments, the blade rack 500 may include a front wall 520 having one or more recesses 522 aligned with respective channels 502, as shown in FIG. 9A. The one or more recesses 522 may function as guides for the corresponding device handle 300 to rest on as the device handle 300 engages with a blade cartridge 400 disposed in the channel 502. That is, a user may rest one end portion of the device head 302 on a recess 522 to align a starting position of the device, and the user may then press down to engage the device head 302 with a corresponding blade cartridge 400.

### Blade Rack Holder

In some embodiments, it may be desirable for the dermaplaning kit to include multiple blade racks 500 in order to keep multiple styles of blades on hand, or to store a larger quantity of blades. It may also be desirable for the dermaplaning kit to include a blade rack holder which may hold multiple blade racks in a compact and organized manner. Even if the dermaplaning kit 100 only includes a single blade rack 500, a blade rack holder configured to hold a single blade rack 500 may still assist in keeping the dermaplaning kit 100 organized and consolidated to allow for easier transport and use of the dermaplaning kit 100.

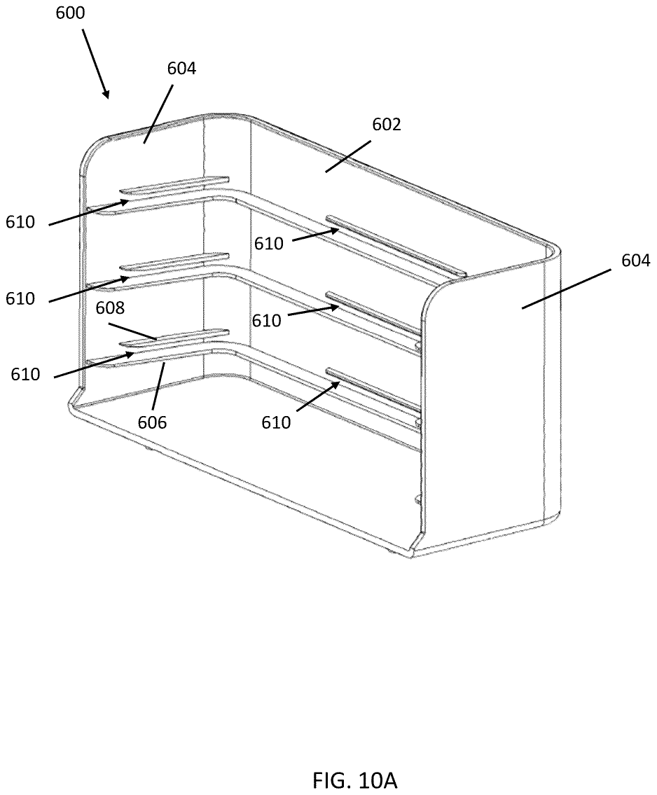

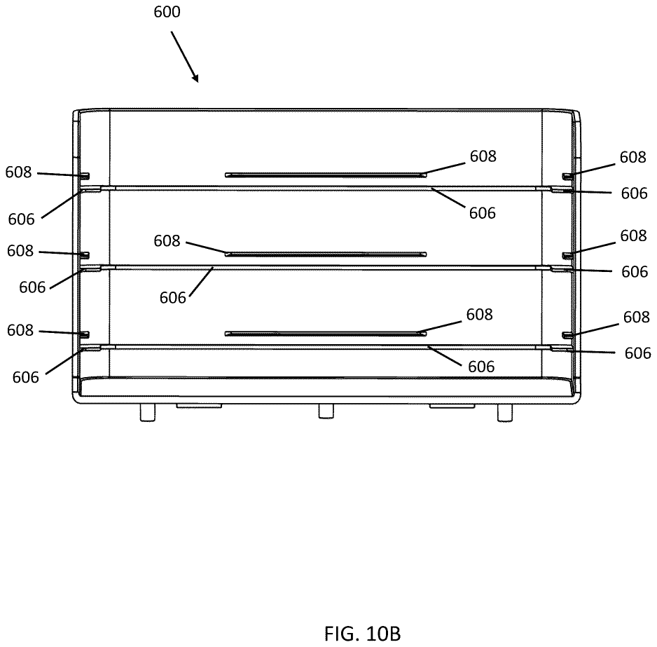

In some embodiments, dermaplaning kit 100 may include a blade rack holder 600 configured to store at least one blade rack 500, as shown in FIGS. 10A-10B. In some embodiments, the blade rack holder 600 may include a back wall 602 and sidewalls 604. In some embodiments, one or more lower protrusions 606 may project from the back wall 602 and sidewalls 604 at a first height. In use, rack support tabs 516 (see FIGS. 9A, 9B, 9C) on blade rack 500 may be placed on the lower protrusions 606, such that the lower protrusions 606 supports the weight of blade rack 500.

In some embodiments, the blade rack holder 600 may include one or more upper protrusions 608 which extend at a second height above and substantially parallel to lower protrusions 606, forming rack channels 610. The blade rack support tabs 516 may be slid into corresponding rack channels 610. The upper protrusions 608 may prevent a blade rack 500 from lifting out of blade rack holder 600 when a blade cartridge 400 is removed from the blade channel 502. In this manner, a user may attach a blade cartridge 400 to device handle 300 and remove the blade cartridge 400 from the blade rack 500 without needing to hold the blade rack 500 in place.

In some embodiments, the rack support tabs 516 and rack channels 610 have an interference fit, so that racks 500 do not fall out of blade rack holder 600. In some embodiments, rack support tabs may have a height similar to a height of the corresponding channel 610 to create an interference fit. In other embodiments, the rack tabs 516 may include tab projections 518 which create the interference fit. The tab projections 518 may include a chamfer 519, which may allow rack tabs 516 to slide smoothly into channels 610. In some embodiments, the tab projections 518 may be constructed and arranged to provide frictional resistance to prevent inadvertent removal of the blade rack 500 from the black rack holder 600. While such an example is disclosed, any suitable gripping or friction features may be provided on the blade rack to prevent movement of the blade rack during use as the disclosure is not so limited.

In some embodiments, the blade rack holder 600 may be configured to hold a single blade rack 500. However, the blade rack holder 600 may be configured to hold as many blade racks as desired. Additional sets of protrusions 606 and 608 forming additional channels 610 may be added to the back wall 602 and sidewalls 604 to accommodate additional blade racks 500. Each set of protrusions may be spaced a sufficient distance to allow for the blade racks 500 to be inserted into blade rack holder 600 without interference from other blade racks.

### Device Receptacle

In some embodiments, it may be desirable for the dermaplaning kit 100 to include a device receptacle 700 to hold device handle 300 when the dermaplaning device 101 is not in use. The device receptacle 700 may include a device groove 702 (also referred to as a device cavity herein) configured to hold the device handle 300. In some embodiments, the device groove 702 may be contoured to match the exterior casing of device handle 300. The device groove may be configured to receive device handle 300 in any orientation. The device receptacle 700 may be configured to receive the handle lengthwise in a longitudinal direction. For example, as illustrated in FIG. 11, the device handle 300 may lay on a side surface in the device groove 702.

In some embodiments, the device groove 702 may be constructed and arranged such that the device handle 300 may slot into the device groove 702 with a blade cartridge 400 attached. In other embodiments, the device groove 702 may be constructed and arranged such that the device handle 300 may only slot into the device groove 702 when no blade cartridge 400 is attached.

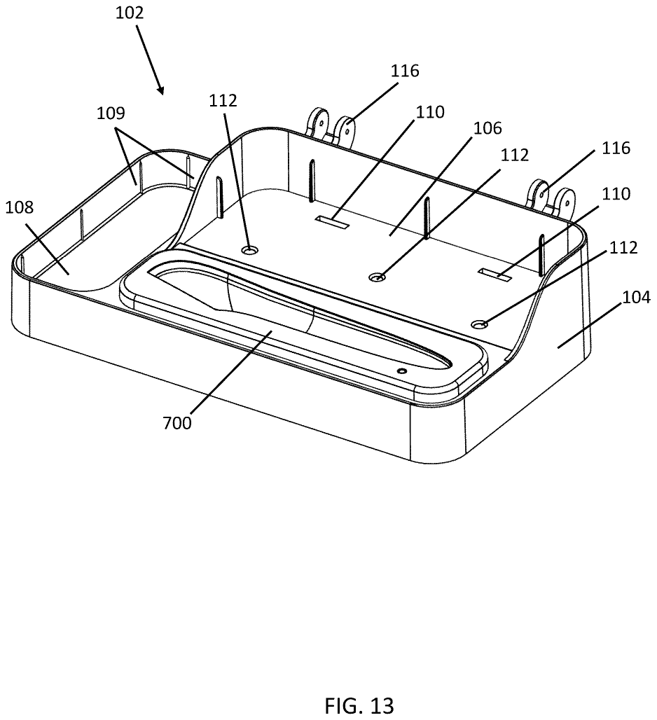

In some embodiments, it may be desirable for the device handle 300 to charge while it is not in use so that a user does not need to remember to recharge the dermaplaning device 101. In some embodiments, the device receptacle 700 may be configured as a charging base and include electrical charging contacts 704 which correspond to electrical charging contacts 320 on the device handle 300. When the device handle 300 is placed in the device groove 702, the charging contacts 704 may contact the charging contacts 320 and allow for the battery to recharge. In some embodiments, the charging contacts 320, 704 may be magnetic to maintain the charging contacts in electrical contact with one another. In some embodiments, the device receptacle 700 may be integrally formed as a part of the base tray 104 (see FIG. 13). In other embodiments, however, the device receptacle 700 may be removably engageable to the base tray 104.

In some embodiments, it may be desirable for the device receptacle 700 to include an indicator light 706 mounted on the device receptacle 700. This indicator light may change in color, brightness, and pattern to provide a user with information about device status. For instance, the indicator light 706 may blink while the device handle 300 is charging and may be solid when the device handle is fully charged. In some embodiments, the indicator light 706 may be a plurality of indicator lights as the disclosure is not so limited.

### Blade Disposal Unit

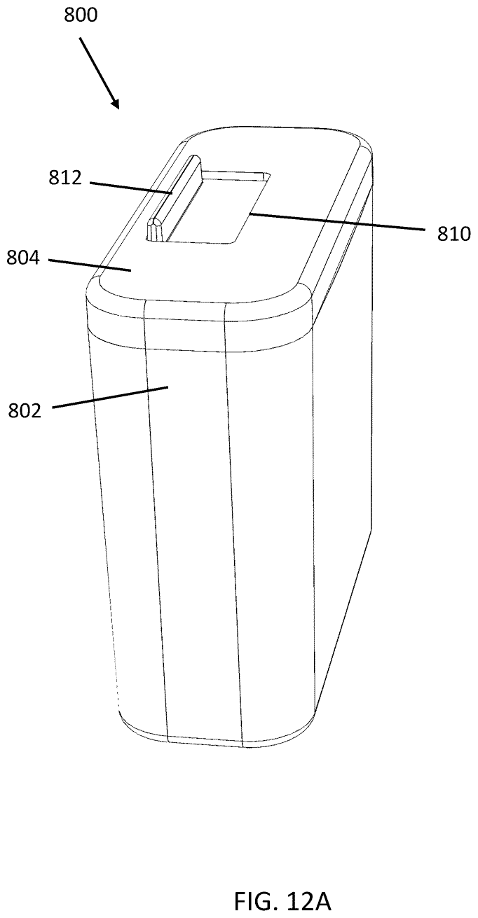

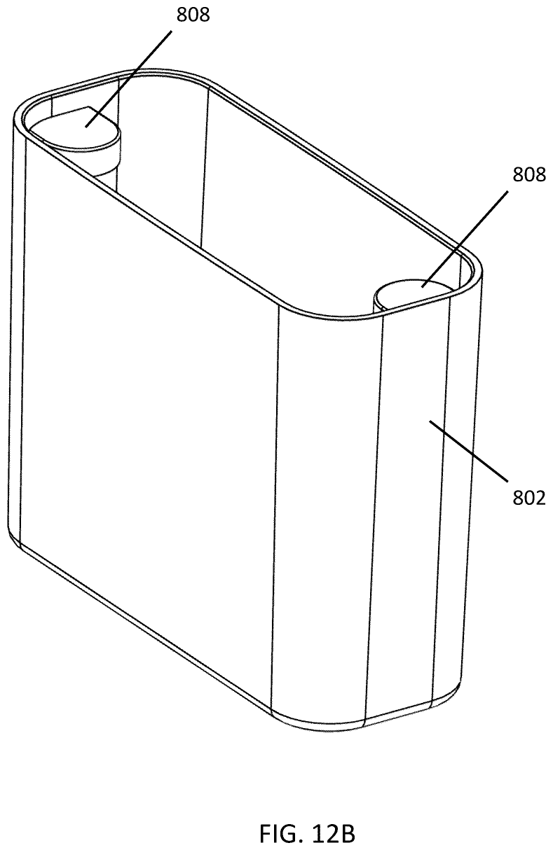

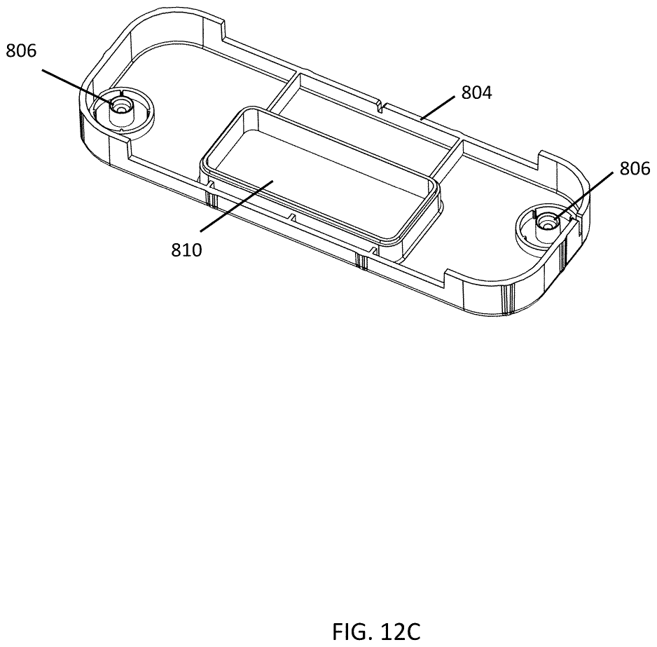

Disposing of used skincare blades may require additional disposal procedures as compared to ordinary trash. Additionally, locations where dermaplaning is performed may not always have proper trash receptacles located within a convenient distance from the user. As shown in FIGS. 12A-12C, the dermaplaning kit 100 may include a blade disposal unit 800 to allow for convenient and efficient disposal of used blade cartridges 400. In some embodiments, the blade disposal unit 800 includes a disposal container 802 configured to hold used or discarded blade cartridges 400.

In some embodiments, the blade disposal unit 800 may include a lid 804 to prevent used blade cartridges from spilling out of the disposal container 802. In some embodiments, the lid 804 may include magnetic fasteners 806 which correspond to magnetic fasteners 808 on the disposal container 802. The magnetic fasteners 806 and 808 may hold the lid 804 onto the disposal container 802, but allow the lid 804 to be easily removed by the user to dispose of the used blade cartridges into a suitable trash receptacle. It should be understood that any suitable fastener may be used to hold the lid 804 to the disposal container 802 such as toggle latches or twist latches. The lid 804 may also be held onto disposal container 802 using an interference fit between the lid and disposal container.

In some embodiments, the lid 804 may include an opening 810 in the lid sized to allow a blade cartridge 400 to drop into the disposal container 802. This may allow a user to dispose of blades without needing to remove the lid 804 from the disposal container 802. In some embodiments, the lid 804 may include a cover 812 configured to selectively cover the opening 810. This may prevent used blade cartridges from falling out of the disposal container 802 if the disposal container is tipped over. The cover 812 may be configured to slide from a closed position, where the opening 810 is covered by the cover 812, to an open position, where the opening 810 is unobstructed by the cover 812.

### Dermaplaning Kit Housing

In some embodiments, it may be desirable for the dermaplaning device 101 and any combination of the peripheral components included in the dermaplaning kit to be consolidated in a kit housing 102. This may allow for easier transport of the entire dermaplaning kit 100, and may help users avoid misplacing peripheral components.

FIG. 13 shows an embodiment of the kit housing 102. In some embodiments, the kit housing 102 may include a base tray 104 (also shown in FIG. 1) configured to hold any combination of the device handle 300 and the peripheral components described above. The peripheral components may be selectively or permanently attached to the base tray using any suitable means. For instance, the peripheral components may be integrally molded with the base tray 104. The peripheral components may also be attached to the base tray 104 using any suitable fasteners such as screws, adhesives, or locking tabs. The peripheral components may also be attached to the base tray 104 using an interference fit.

The base tray 104 may also include a blade rack holder attachment area 106 and a disposal unit attachment area 108. The blade rack holder 600 and the disposal unit 800 may be attached to these respective attachment areas using any suitable fastening methods such as those described above.

In some embodiments, it may be desirable for the kit housing 102 to include a housing lid 114, as shown in FIG. 1. The housing lid 114 may be configured to selectively cover any of the components in the dermaplaning kit 100. The housing lid 114 may protect various components from falling objects. It may also limit the amount of dust which contacts the various components. The housing lid 114 may also improve the overall aesthetic appearance of the dermaplaning kit 100. In some embodiments, the housing lid 114 may be attached to the base tray 104 using hinges 116. It should be understood that any fasteners may be used to attach the lid 114 to the base tray 104, such as latches or by interference fit, as the disclosure is not so limited.

In one embodiment, the housing lid 114 is configured to cover the device handle 300, the blade cartridges 400, the blade racks 500, the blade rack holder 600, and the device receptacle 700. It should be understood that housing lid 114 may be configured to cover any combination of components.

### Blade Cartridge Edge Design

FIG. 14 shows an embodiment of the device head 302 engaged with a blade cartridge 400, where the device is being oriented with respect to a user's skin 900 at an angle of access “AA”. As shown in FIG. 14, the blade cartridge, and more specifically the first and second blade housing sections 414, 416, are curved inwardly towards the edge of the blade such that the device head 302 is able to approach the skin 900 at a lower angle of access in comparison to a cartridge housing that is not curved towards the edge of the blade. The inventors have appreciated that a lower angle of access may be beneficial to remove vellus hair from the user's skin. In some embodiments, the blade cartridge may be arranged such that the angle of access AA during use is greater than or equal to 5°, 10°, 15°, 20°, 25°, 30°, 35°, 40°, 45°, 50°, 55°, or greater. Likewise, in some embodiments, the blade cartridge may be arranged such that the angle of access AA during use is lesser than or equal to 60°, 55°, 50°, 45°, 40°, 35°, 30°, 25°, 20°, 15°, 10°, or lesser. Of course, values both greater or lesser than those disclosed above are also contemplated and may be selected according to the degree of curvature of the blade housing sections.

### Method of Use

The following is an exemplary embodiment of a method of using the dermaplaning kit 100. When not in use, the device handle 300 may be placed in the device receptacle 700 and be charged via the charging contacts 320 and 704. To use the dermaplaning device 101, the lid 114 may first be moved to an open position. The device handle 300 may then be removed from the device receptacle 700. A blade cartridge 400 may then be attached to the device handle 300, and the power button 316 on the device handle 300 may be used to set the desired vibration mode. The dermaplaning may be performed by sweeping blade 402 across the skin. Once dermaplaning is completed, the cover 812 on blade disposal unit 800 may be opened to reveal opening 810. The blade cartridge 400 may then be held over opening 810, and the button 312 may be depressed to eject blade cartridge 400 from the device handle 300 and into the disposal container 802. Cover 812 may then be closed, and device handle 300 may be returned to the device receptacle 700. The lid 114 may then be returned to a closed position to cover the components.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A dermaplaning kit comprising:
a base tray;
a charging base configured to receive and charge a dermaplaning device;
a blade cartridge storage rack supported by the base tray, the blade cartridge storage rack configured to hold a plurality of blade cartridges for attachment to the dermaplaning device; and
a blade cartridge disposal unit supported by the base tray, the blade cartridge disposal unit configured to receive and hold discarded blade cartridges.

2. The dermaplaning kit of claim 1, wherein the charging base is integrally formed with the base tray.

3. The dermaplaning kit of claim 1, wherein the charging base includes a charging cavity configured to receive and charge the dermaplaning device, wherein the charging cavity has a shape corresponding to a shape of the dermaplaning device.

4. The dermaplaning device of claim 3, wherein the charging cavity includes one or more electrical contacts which align with one or more electrical contacts on a handle of the dermaplaning device when the dermaplaning device is placed in the charging cavity.

5. The dermaplaning kit of claim 1, wherein the base cartridge disposal unit includes a disposal container and a lid engaged with the container, the lid having an opening configured to receive and hold the discarded blade cartridges.

6. The dermaplaning kit of claim 5, wherein the lid further includes a movable cover configured to move between an open position in which the opening is covered by the cover, and a closed position in which the opening is unobstructed by the cover.

7. The dermaplaning kit of claim 1, wherein the blade cartridge storage rack includes a plurality of channels each configured to hold a blade cartridge of the plurality of blade cartridges.

8. The dermaplaning kit of claim 7, wherein each of the plurality of channels include one or more tabs configured to engage one or more corresponding grooves in a respective blade cartridge of the plurality of blade cartridges to retain the blade cartridges in the corresponding channels.

9. The dermaplaning kit of claim 8, wherein the one or more tabs are compliant such that each of the blade cartridges received in the corresponding channels are removable from the channels in response to a threshold force being exerted on the one or more tabs.

10. The dermaplaning kit of claim 7, wherein the blade cartridge storage rack includes a wall having a plurality of recesses, wherein each of the recesses are aligned with a corresponding channel of the plurality of channels such that the recesses are arranged to receive and align the dermaplaning device for attachment to a corresponding blade cartridge of the plurality of blade cartridges.

11. A dermaplaning kit comprising:
a dermaplaning device including a handle and at least one blade cartridge attachable to the handle, the handle extending lengthwise in a longitudinal direction and including a head at one end portion thereof, the head configured to attach the at least one blade cartridge to the handle, the device configured to generate vibrations at the head; and
a charging base including a charging cavity configured to receive and charge the dermaplaning device,
wherein the charging cavity has a shape corresponding to a shape of the dermaplaning device, and wherein the charging cavity includes one or more electrical contacts which align with one or more electrical contacts on the handle when the dermaplaning device is placed in the charging cavity.

12-16. (canceled)

17. A dermaplaning kit comprising:
a base tray configured to receive and store a dermaplaning device; and
a blade cartridge rack holder supported by the base tray, the blade cartridge rack holder configured to hold a plurality of blade cartridge storage racks, each blade cartridge storage rack configured to hold a plurality of blade cartridges for attachment to the dermaplaning device.

18-22. (canceled)

23. A dermaplaning device comprising:
a blade cartridge including a blade;
a handle extending lengthwise in a longitudinal direction, the handle including a head at a first end portion thereof which is configured to releasably attach the blade cartridge to the handle;
a catch movable between a locked position and an unlocked position, the catch configured to retain the blade cartridge at the head when moved to the locked position and to release the blade cartridge from the head when moved to the unlocked position; and
an actuator coupled to the catch and configured to move the catch to the unlocked position upon actuation of the actuator.

24-33. (canceled)

34. A blade cartridge for a dermaplaning device, the blade cartridge comprising:
a blade;
a housing attached to the blade; and
a safety cage configured to be attached to the blade, the safety cage being configured to limit a depth at which the blade contacts a user's skin,
wherein the safety cage comprises a plurality of projections which extend from a side of the blade to an edge of the blade, and wherein the plurality of projections each have a tapered shape such that a width of a proximal portion of the projections is greater than a width of a distal portion of the projections.

35-46. (canceled)

47. A blade cartridge for a dermaplaning device, the blade cartridge comprising:
a blade;
a housing attached to the blade; and
a mounting flange including a first pair of latches configured to engage a corresponding second pair of latches on a dermaplaning device to attach the blade cartridge to the dermaplaning device.

48. The blade cartridge of claim 47, wherein the first pair of latches is hook-shaped.

49. The blade cartridge of claim 47, wherein each latch of the first pair of latches includes a first cam surface and each latch of the second pair of latches includes a second cam surface, the first cam surfaces cooperating with the second cam surfaces to attach the blade cartridge to the dermaplaning device.

50-53. (canceled)

54. The blade cartridge of claim 47, in combination with the dermaplaning device, the dermaplaning device including a handle with a head at a first end portion of the handle, wherein the head is configured to releasably attach the blade cartridge to the handle through an aperture in the head.

55. The blade cartridge of claim 54, wherein the aperture has a first region and a second region of a greater width than the first region, and wherein the mounting flange of the blade cartridge is configured to extend into the aperture to attach the blade cartridge to the handle.

56. The blade cartridge of claim 47, wherein the mounting flange has a first region and a second region of different width than the first region such that the blade cartridge is configured to be attached to the dermaplaning device only when the first and second regions of the mounting flange are aligned with respective regions of corresponding width on the dermaplaning device.
